(19) United States
(12) Patent Application Publication
(10) Pub. No.: US 2006/0269623 A1
(43) Pub. Date: Nov. 30, 2006

(54) Herbal composition and method of use for promoting erections and treating erectile dysfunction in men

(76) Inventor: Pierre Swaab, Amsterdam (NL)

Correspondence Address: ERIC HANSCOM, 7395 PORTAGE WAY, CARLSBAD, CA, 92011, US

(21) Appl. No.: 11/140,141

(22) Filed: May 31, 2005

(51) Int. Cl. A61K 36/54; A61K 36/254; A61K 36/10; A61K 36/704

(52) U.S. Cl. 424/728; 424/762; 424/739; 424/773

(57) ABSTRACT

This invention is directed toward herbal compositions and methods of treatment for prevention or treatment of erectile dysfunction disorders and ameliorating symptoms thereof and as a preventative measure against erectile dysfunction. The methods comprise administering a therapeutically effective composition of matter comprising the following herbal and other components: Herba cynomorii, Rhizhomnas atractylodis macrocephalae, Radix rehmannia glutinosea longui, Herba epimedii, Fructus lycii, Fructus schisandrae chinensis, Radix poloygoni multiflor, Cortex cinnamonia cassiae, Fructus amoni, and Radix ginseng. The components of the invention interact synergistically once consumed to remedy or prevent erectile dysfunction when taken as prescribed. The invention is completely herbal in composition and does not contain any hormones, morphine, or any of the compositions of matter know by trade names Viagra, Cialis, Sildenafil or Vardenafil. Side benefits of taking this dietary supplement include increased energy, improved immune system strength, and improved erectile activity in men without erectile dysfunction.

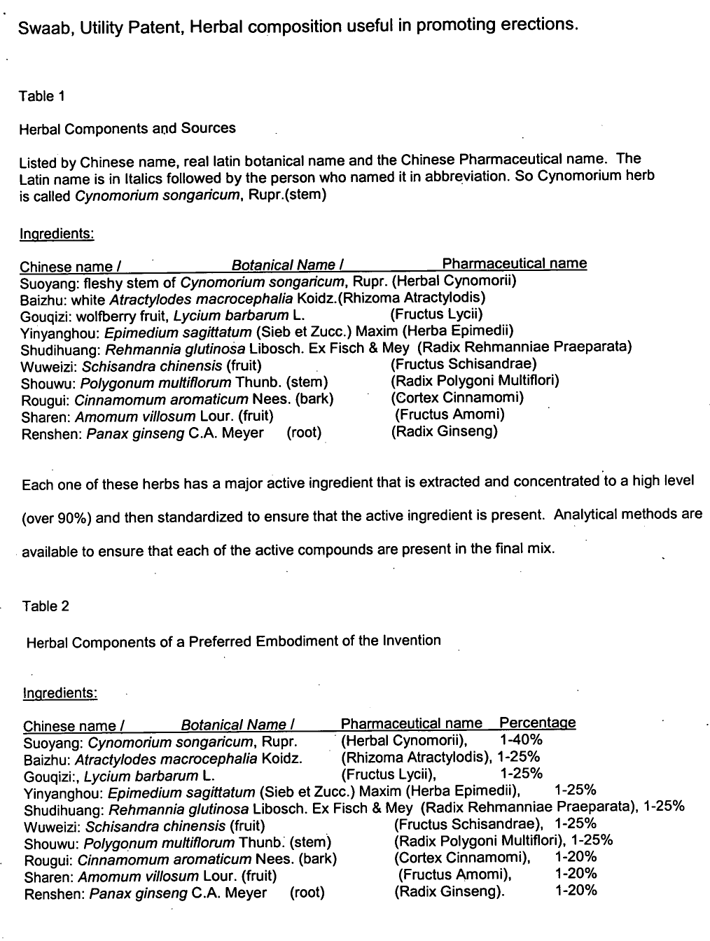

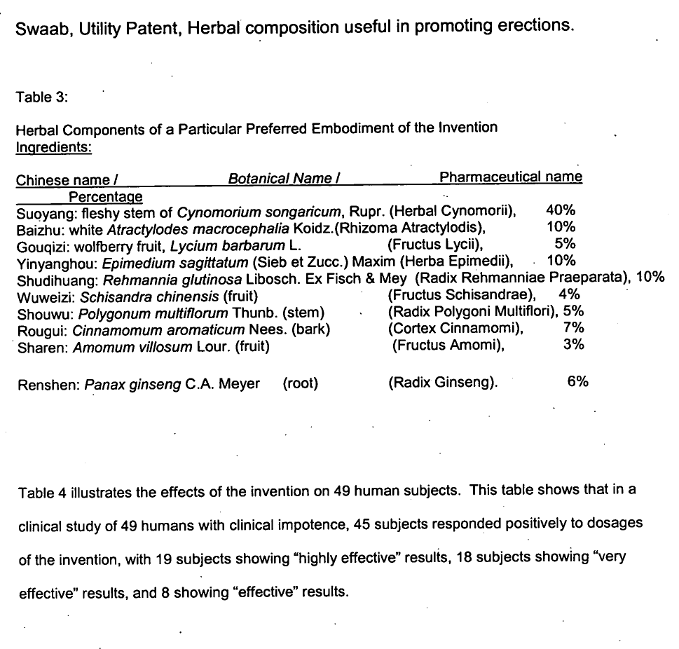

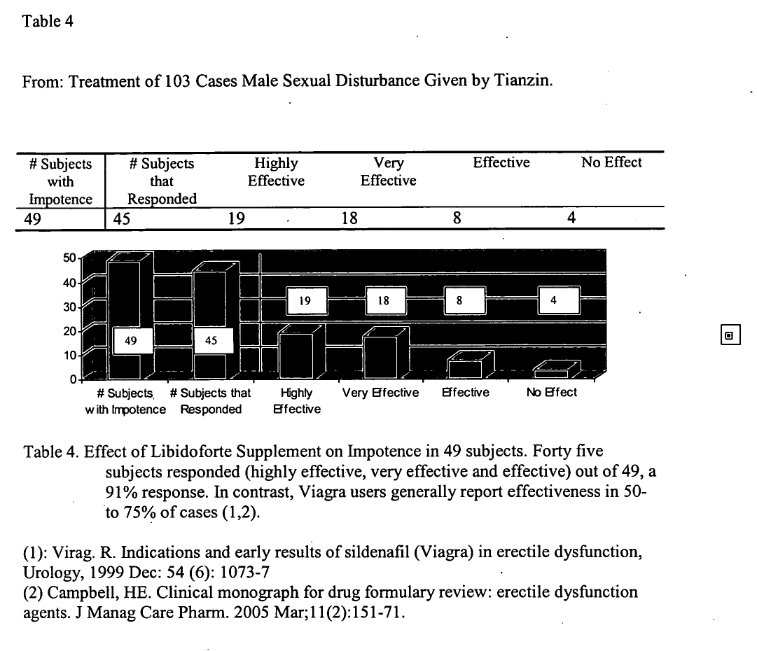

### CROSS REFERENCE TO RELATED APPLICATIONS

None.

### STATEMENT REGARDING FEDERALLY SPONSORED RESEARCH OR DEVELOPMENT

This invention was not federally sponsored.

### BACKGROUND OF THE INVENTION

This invention is directed toward herbal compositions and methods of treatment for prevention or treatment of erectile dysfunction disorders in normal men and ameliorating symptoms thereof and as a preventative measure against erectile dysfunction, where the herbal composition has related side health benefits. The methods comprise administering a therapeutically effective composition of matter comprising the following herbal and other components: Herba cynomorii, Rhizhomnas atractylodis macrocephalae, Radix rehmannia glutinosea longui, Herba epimedii, Fructus lycii, Fructus schisandrae chinensis, Radix poloygoni multiflor, Cortex cinnamonia cassiae, Fructus amoni, and Radix ginseng. The components of the invention interact synergistically once consumed to remedy or prevent erectile dysfunction when taken as prescribed. The invention is completely herbal in composition and does not contain any hormones, morphine, or any of the compositions of matter know by trade names Viagra, Cialis, Sildenafil or Vardenafil. Side benefits of taking this dietary supplement include increased energy, improved immune system strength, and improved erectile activity in men without erectile dysfunction.

Drugs derived from plants have been part of humankind's efforts to treat medical problems for thousands of years. Currently, many thousands of drugs, both those commonly used and prescribed and those used only in extremely rare instances were derived from a plant source, have at least part of their composition derived from a plant source, or contain chemical imitations of a plant compound. Many of the drugs used today contain herbal components and/or herbal extracts. Botanically, an herb can be described as a small (generally), fleshy stemmed, non-woody, annual or perennial seed-bearing plant in which all the above-ground parts die back at the end of each growing season, which usually coincides with the arrival of fall or spring. As used with respect to alternative medicines, the word “herb” refers to a plant or plant part which has a medicinal use, including the seeds, leaves, stems, flowers, roots, berries, bark, or any other plant parts that are used for healing. Dietary supplements such as the current invention are widely available through general retail stores, health food stores, grocery stores, pharmacies, by mail, and over the internet.

Prior to the passage of the Dietary Supplement Health and Education Act of 1994 (DSHEA), the term “dietary supplement” referred to edible products comprising one or more of the essential nutrients, such as vitamins, minerals, and protein. The DSHEA broadened the phraseology to include most products produced to be taken orally as a supplement to a human's diet, including minerals, vitamins, herbs, botanicals, and other plant-derived substances; and amino acids and concentrates, metabolites, constituents and extracts of these substances. Dietary supplements are generally not represented as a conventional food or as a sole item of a meal or of the diet (Dietary Supplement Health and Education Act of 1994, Section Three), and are usually made for consumption in the form of a coated or uncoated pill, capsules with a water permeable cover protects a granular or powdered composition from being immediately released into the user's mouth, tablets, liquids, powders, soft gels.

The DSHEA in addition to redefining the term “dietary supplement” set forth new regulations covering dietary supplements (Dietary Supplement Health and Education Act, Public L. No. 103-417, 108 Stat. 4325 (1994)). Specifically, the DSHEA allows manufacturers to dietary supplements to make limited statements regarding the nutritional benefits of their products, including statements that: (1) claims a benefit to a classical nutrient deficiency disease and discloses the prevalence of such disease in the U.S.; (2) describes the role of a nutrient or dietary ingredient intended to affect the structure or function in humans; (3) characterizes the documented mechanism by which a nutrient or dietary ingredient acts to maintain such structure or function; or (4) describes general well-being from consumption of a nutrient or dietary ingredient (Dietary Supplement Health and Education Act, Public L. No. 103-417 .sctn. 6, 108 Stat. 4325, 4329 (1994)).

The medical situation addressed by this invention or dietary supplement, is erectile dysfunction, a new term for what was previously referred to as impotence. In medical terms, erectile dysfunction is the inability to obtain and maintain and erection that is firm enough for penetration by the man during intercourse, and/or the ability to keep the erection long enough to ejaculate inside the vagina. A man is considered to have erectile dysfunction if he is unable to obtain or maintain an erection in at least 50 percent of his attempts at intercourse.

Let us turn briefly to how erectile dysfunction operates, or, perhaps, cause the erection to fail to operate depending on the perspective taken. A man's erection is dependant upon two chambers, technically referred to as corpora cavernosa, which run the length of the penis, which are filled with spongy tissue. Surrounding the corpora cavernosa is a membrane called the tunica albuginea. The spongy tissue inside the corpora cavernosa a number of items, including smooth muscles, fibrous tissues, spaces, veins, and arteries. The urethra, which is the channel for both urine and ejaculate, runs beneath the corpora cavernosa.

Penile erection is initiated by one or both of sensory or mental stimulation, whereupon the the muscles of the corpora cavernosa are relaxed by neural impulses from the brain and local nerves. This relaxation causes blood to flow into the corpora cavemosa and fill the spaces, creating elevated blood creates pressure, making the penis expand. The blood in the corpora cavemosa is then partially trapped by the tunica albuginea, thereby sustaining the man's erection. The erection is reversed when penile muscles contract to stop the inflow of blood and open outflow channels. Erectile dysfunction is basically a failure in any link of the chain toward obtaining and maintaining an erection. Since erections require a particular sequence of events, erectile dysfunction can occur when any single one off the events in the chain is disrupted. Erectile dysfunction is often caused by physical damage to key organs and systems involved in the erective process. The most common cause of erectile dysfunction is damage to nerves, arteries, smooth muscles, and fibrous tissues, often as a result of diseases, such as diabetes, kidney disease, chronic alcoholism, multiple sclerosis, atherosclerosis, vascular disease, and neurological disease. For example, between 35 and 50 percent of men with diabetes experience erectile dysfunction. Additionally, several types of surgery (especially radical prostate and bladder surgery for cancer) can injure nerves and arteries near the penis, causing erectile dysfunction. Finally, physical injury to the penis, spinal cord, prostate, bladder, and pelvis can lead to ED by harming nerves, smooth muscles, arteries, and fibrous tissues of the corpora cavernosa.

In addition to the physical causes of erectile dysfunction, many common medicines, such as drugs for elevated blood pressure, antihistamines, antidepressants, tranquilizers, appetite suppressants, and cimetidine (an ulcer drug), can, in some men, produce erectile dysfunction as a side effect. As erectile dysfunction becomes a more recognized and treated medical condition, it is expected that the number of common, household medicines that cause erectile dysfunction as a side effect will grow. Other possible physical causes are smoking, which affects blood flow in veins and arteries, and hormonal abnormalities, such as a genetic lack of testosterone. Erectile dysfunction also has a substantial psychological component. Many experts contend that emotional factors such as stress, anxiety, guilt, depression, low self-esteem, and fear of sexual failure cause 10 to 20 percent of ED cases. Men with a physical cause for ED frequently experience the same sort of psychological reactions (stress, anxiety, guilt, depression), creating a sort of “Catch-22”, where even had they entered the sexual arena without psychological problems, their physical problems which cause the erectile dysfunction may lead to the psychological problems which only reinforce their concerns over erectile dysfunction. With most men, erectile dysfunction does not diminish their sex drive, nor impair their ability to ejaculate once they have achieved an erection and penetration. Erectile dysfunction is a medical condition with a variety of potentially serious emotional side effects for both the man and his partner. While some cases of erectile dysfunction are temporary and will disappear, at least temporarily, without medical treatment, other cases are more severe and require intervention.

While erectile dysfunction as been an unfortunate human condition for thousands of years, the frequency of it being seen as a treatable medical condition has only become widely known in the non-medical and non-scientific communities during the last few years due to the greatly increased number of advertisements for erectile dysfunction treatments. Indeed, recent surveys have shown the erectile dysfunction treatments have replaced even pornography as the most frequent type of internet spam, which provides an annoying, but fairly accurate barometer of the popularity of various erectile dysfunction products for sale over the internet.

The frequency of erectile dysfunction has been shown in case studies to be fairly high. Indeed, up to one third of all men and over a half of all adult men over 50 have been affected by erectile dysfunction. While erectile dysfunction can strike a man at any age, his chances of experiencing erectile dysfunction increase with age, particularly after the age of 50. Regardless of the cause of the erectile dysfunction, erectile dysfunction can decrease a man's self esteem and harm his relationship with his partner. Because erectile dysfunction is still seen by many as an embarrassing “personal problem” and not a treatable medical condition, it is likely that even these numbers are on the low side.

The prior has several examples of attempts to resolve this problem.

1. Herbal Treatments for Erectile Dysfunction.

There are a number of herbal treatments for erectile dysfunction, but the past and current herbal treatments present many problems.

One problem with many herbal formulas is that they take effect too slowly, with poor stability and a lack of standardization. This may be due to imperfect extraction techniques, or improper mixing of the ingredients in terms of method or percentages.

Another problem with the current herbal treatments is the relative lack of knowledge about the active ingredients. Many herbal products contain a multitude of ingredients and the major active ingredients have not been thoroughly researched and studied, hence there can be tremendous “lot to lot” variability with respect to the active ingredients in terms of their state and function.

An addition difficulty is the current lack of standardization of the active ingredients. Even if the active ingredients are well known, many herbal treatments are not standardized with respect to the active ingredient and the characteristics of the ingredient as it is harvested from natural material: This results, again, with the potential for lot to lot differences which, in turn, creates differences in the effects each lot produces.

Furthermore, many herbal ingredients have not been tested in standardized clinical trials either invivo or invitro. Many current sellers of herbal treatments rely more upon the “Folklore” aspects of the herb than upon the scientific basis when formulating a treatment for a particular disorder. For example, “Horny Goat weed” is a frequent ingredient of erectile dysfunction herbal treatments, based almost entirely upon the folklore of goats becoming aroused after consuming the herb. It has not been tested in humans for such an effect nor is there sound scientific proof that it even works on goats.

Past and current herbal treatments are also plagued by the fact that some herbal treatments are standardized to an ingredient that may not be the active ingredient at all. Using Horny Goat weed as an example, Horny Goat weed-containing herbal treatments are frequently standardized to its icariin content, but many scientists do not believe that icariin is the real active ingredient at all.

Some herbal compounds have severe side effects as well. The best example is Yohimbe, an herb from the bark of an African tree. It has some libido-enhancing activity but also causes severe anxiety in many who take it, along with higher blood pressure.

Finally, herbal treatments may also be contaminated with drugs or heavy metals. Many herbal treatments have been spiked with Viagra or another erectile dysfunction compound, some herbal treatments are found to contain high levels of either pesticides or heavy metals.

1. Non-Herbal Treatments for Erectile Dysfunction.

There are a number of non-herbal treatments for erectile dysfunction, many of which are heavily advertised products that appear in the print media as well as on television and radio advertisements and the internet.

Currently, there are three oral agents approved for use by the Food and Drug Administration (FDA). These are Sildenafil Citrate (Viagra approved March 1998) and Vardenafil Hydrochloride (Levitra approved September 2003) and Tadalafil (Cialis approved November 2003).

All three drugs reversibly inhibit the penile-specific phosphodiesterase (type 5; PDE-5) and enhance the nitric oxide-cyclic GMP pathway of cavernous smooth muscle relaxation (i.e., all three prevent breakdown of cyclic GMP by PDE-5). In several double-blind, placebo-controlled studies of patients with erectile dysfunction of varied etiology, all three drugs demonstrated improvement in erectile function, with success rates varying between 70% and 90% depending on the populations studied.

Side effects of all three include headache, flushing, dyspepsia, and nasal congestion. Visual abnormalities are encountered with sildenafil, but are less likely with vardenafil and unlikely with tadalafil. Back pain and myalgia may occur with tadalafil, but are unusual with either sildenafil or vardenafil. All three drugs are contraindicated in patients who use nitroglycerin or nitrate-containing compounds. Combining any of these three drugs with nitroglycerin or nitrates may result in significant hypotension.

Thus there has existed a long-felt need for a treatment for erectile dysfunction which both works effectively and does not carry with it undesirable side effects such as stuffy nose or nasal congestion, upset stomach, especially when taken with food, lowered blood pressure, significant visual streaking or ghosting of light, bluish tinge to white lights, headaches, facial flushing, diarrhea, dizziness and rashes.

The current invention provides just such a solution by providing herbal compositions and methods of treatment for prevention or treatment of erectile dysfunction disorders and ameliorating symptoms thereof. The methods comprise administering a therapeutically effective composition of matter comprising the following herbal and other components: Herba cynomorii, Rhizhomnas atractylodis macrocephalae, Radix rehmannia glutinosea longui, Herba epimedii, Fructus lycii, Fructus schisandrae chinensis, Radix poloygoni multiflor, Cortex cinnamonia cassiae, Fructus amoni, and Radix ginseng.

The components are obtained through traditional Chinese herbal methods and more modem techniques, including some combination of one or more of the following factors: boiling in water or other solvents-such as alcohol, acids and bases, distilling an extract and boiling to concentrate the extract, grinding up an herb with a mortar and pestle, drying an herb in the sun or with a mechanical drying machine, washing, rinsing, and spin-drying an herb, straining, evaporation and the use of a desiccation chamber. Other forms are contemplated, but it is desired to have aqueous or alcohol and aqueous extracts of each herb. It is contemplated that the herbs will be dried individually, or in mixtures, such as whole dried plants or plant parts, or powders of ground and dried plants or plant parts, or powders or extracts or decoctions of plants and other components.

In a preferred embodiment, the composition comprises an extract of the following components in the approximate percentages, shown as ranges, as dry weight: Herba cynomorii (35-45%), Rhizhomnas atractylodis macrocephalae (8-13%), Radix rehmannia glutinosea longui (8-13%), Herba epimedii (8-13%), Fructus lycii (3-8%), Fructus schisandrae chinensis (3-7%), Radix poloygoni multiflor (3-7%), Cortex cinnamonia cassiae (5-10%), Fructus amoni (2-5%), and Radix ginseng (4-8%), with or without further comprising a pharmaceutically acceptable carrier, diluent or additive.

In a particularly preferred embodiment, the composition comprises an extract of the following components in the approximate percentages, as dry weight: Herba cynomorii (40%), Rhizhomnas atractylodis macrocephalae (10%), Radix rehmannia glutinosea longui (10%), Herba epimedii (10%), Fructus lycii (5%), Fructus schisandrae chinensis (4%), Radix poloygoni multiflor (5%), Cortex cinnamonia cassiae (7%), Fructus amoni (3%), and Radix ginseng (6%) with or without further comprising a pharmaceutically acceptable carrier, diluent or additive.

The components of the invention interact synergistically once consumed to remedy or prevent erectile dysfunction when taken as prescribed. The invention is completely herbal in composition and does not contain any hormones, morphine, or any of the compositions of matter know by trade names Viagra, Cialis, Sildenafil or Vardenafil. Side benefits of taking this dietary supplement include increased energy, improved immune system strength, and improved erectile activity without erectile dysfunction. The invention has no side effects, is not toxic, and is non-addictive.

The invention is the culmination of thousands of years of Chinese herbal medicine combined with and improved by modern methods of extracting key ingredients and components from herbs. Traditional Chinese herbalists have been treating erectile dysfunction for thousands of years with combinations of herbs. According to Traditional Chinese Medicine, erectile dysfunction is caused by functional disorders or degeneration of the zangfu organs, which is caused mainly by a deficiency of the kidney and the disharmony of the spleen and kidney, and accompanied with blood stasis and phlegm turbidity. Under the TCM theory, the invention theoretically reinforces qi, tonifies yang; activates blood, and removes obstructions of meridians. Because this invention combines the use of extracts of known medicinal benefit with greatly improved methods of extraction, it performs substantially better than previous herbal attempts at remedying erectile dysfunction.

The invention is not only different from and performs superior to other herbal supplements, but it is also very different in composition and theoretical basis from Viagra, Cialis, and other popular western prescription and non-prescription substances for the treatment of erectile dysfunction. In terms of its composition, the invention does not contain any of the common western compositions, including Sildenafil, Cialis, and Vardenafil. This has been proven by a combination of testing methods, including HPLC, TLC, Gas Chromatography, thin-layer chromatography, IR, and UV, administered by scientists at Northwest University, China Academy of TCM, the Medicinal Herb Institute of China Academy of Medical Science and Quinghua University.

Safely Assessment of the Invention.

A safety assessment was performed on the invention by the Chinese Province Station: Department of Epidemic Prevention and the authority for the Department of Health Products “Experimental Appraisal for Health Products Province”. Their clinical tests showed:

1. Acute Toxicity (LD 50) using the Chinese “Big Mouse” species specifically used to extrapolate the LD 50 dosages to humans. “Big Mouse” is the name of a species of mice that are larger than normal in body weight. It is also called the Kunming mice strain commonly used by the Beijing Municipal Center for Hygiene and Epidemic Control. The LD50 (amount of the invention that needed to be applied such that 50% of the test animals died from the “lethal dose”) was greater than 21.6 g/kg.bw (I assume this is 21.6 grams per kilogram of body weight)), which is a does 2,160 times the dosage recommended for the human body.

1. A micronucleus test of the marrow cell of a mouse gave a negative result. This test is a standard in vitro test that measures the toxicity of a compound via induction of micronuclei generated by the red marrow cell of a mouse. Compounds that are toxic induce many small nuclei that can be counted. This test that no extra nuclei are formed and that in this test the compounds are not toxic or mutagenic.

Here is an exact method or test that was used. Mice marrow polychromatic erythrocytes micronucleus test: This test was done by oral administration in two times with 24 h interval. White mice 50 with weight 25-30 g were divided into five groups randomly according to body weight, 10 mice in each group with 5 female and male respectively. Cytoxan with dose of 50 mg/kgi¤b.w was set as positive control group and distilled water as negative control group. Enwei Wellcare Tablet with dose of 2.5, 5.0 and 10.0 g/kgi¤b.w were soluted with distilled water into needed density. 6 h later from the last administration of Enwei Welicare Tablet, killing the mice by ceruical uertebra dislocation, taking chest marrow and diluting smear with calf serum, fixing with methanol and dyeing with Giemsa. Taking 1000 polychromatic erythrocytes (PRC) for each mouse, micronucleus incidence rate was calculated on the base of PRC containing micronucleus in 0/00.

2. A test for deformity of mouse sperm was negative. This test was performed as follows. The sexual matured mice 50 with weight 30-35 g were divided into five groups randomly. Cytoxan with dose of 40 mg/kgi¤b.w was set as positive control and distilled water as negative control. Enwei Wellcare Tablet with dose of 2.5, 5.0 and 10.0 g/kgi¤b.w were soluted with distilled water into needed density, one time each day by oral administration to the mice for 5 days. Killing mice 30 days later from last administration, taking epididymides to prepare smear, dyeing with eosin. Taking 5 mice in each group and counting 1000 well-structured spermatozoa for each mouse, calculating incidence rate of spermatozoa deformity in percentage.

3. The Ames test, named for its developer, Bruce Ames, a test for determining if a chemical is a mutgen, was negative. The use of the Ames test is based on the assumption that any substance that is mutagenic (for the bacteria used in his test) may also turn out to be a carcinogen; that is, to cause cancer. Although, in fact, some substances that cause cancer in laboratory animals (dioxin, for example) do not give a positive Ames test (and vice-versa), the ease and low cost of the test make it invaluable for screening substances in our environment for possible carcinogenicity.

4. There were no abnormalities found on the index of mice raised under dosages of the invention at 2.5 g/kg.bw-10 g/kg.bw for 30 days, (this dosage corresponds to 1,000 times the dosage recommended for the human body).

Teratogenicity tests on mice did not show any teratorgenisis between 2.3 g/kg.bw-1.0 g/kg.bw.

### SUMMARY OF THE INVENTION

It is a principal object of the invention to provide a safe and effective treatment for erectile dysfunction.

It is another object of the invention to provide a preventative measure that a user can take to prevent erectile dysfunction.

It is an additional object of the invention that the key components of this invention be extracted in a way that enhances their concentration and purity, which thereby enhances their effectiveness.

It is a further object of the invention to provide herbal compositions and methods of treatment for prevention or treatment of erectile dysfunction disorders and ameliorating symptoms thereof.

It is also an object of this invention to provide a treatment for erectile dysfunction comprising the following herbs and herbal extracts: Herba cynomorii, Rhizhomnas atractylodis macrocephalae, Radix rehmannia glutinosea longui, Herba epimedii, Fructus lycii, Fructus schisandrae chinensis, Radix poloygoni multiflor, Cortex cinnamonia cassiae, Fructus amoni, and Radix ginseng.

It is another object of the invention to provide a treatment for erectile dysfunction in which the components of the invention interact synergistically once consumed to remedy or prevent erectile dysfunction when taken as prescribed.

It is yet a further object of the invention that the components of the invention be prepared from the leafs, flowers, stems, bark and roots of various herbs.

It is-an additional object of the invention to provide a treatment for erectile dysfunction in which the invention is completely herbal in composition

It is also an object of the invention to provide a treatment for erectile dysfunction in which the invention does not contain any hormones, morphine, or any of the compositions of matter know by trade names Viagra, Cialis, Sildenafil or Varenifil.

It is a further object of the invention to provide a method by which a person with erectile dysfunction can be treated through the use of this invention where the invention has no side effects, is not toxic, and is non-addictive.

It is also an object of this invention to provide a method by which a person with erectile dysfunction can be treated through the use of this invention by the self-administration of a therapeutically effective composition of matter.

It is an additional object of this invention to provide a substance and a method by which a non erectile dysfunction-impaired individual can use the substance as a preventative measure against potential future onsets of erectile dysfunction.

It is a final object of this invention to provide additional side benefits to users of the invention, including increased energy, improved immune system strength, and improved erectile activity without erectile dysfunction.

It should be understood the while the preferred embodiments of the invention are described in some detail herein, the present disclosure is made by way of example only and that variations and changes thereto are possible without departing from the subject matter coming within the scope of the following claims, and a reasonable equivalency thereof, which claims. I regard as my invention.

### BRIEF DESCRIPTION OF THE FIGURES

Table 1 lists the components of the invention.

Table 2 lists the components of the invention in a preferred embodiment of the invention showing the preferred proportions.

Table 3 lists the components of the invention in a particularly preferred embodiment of the invention showing the particularly preferred proportions.

Table 4 illustrates the effects of the invention on 49 human subjects.

### DETAILED DESCRIPTION OF THE INVENTION

The invention is based upon a combination of ancient Chinese medicinal uses of herbs combined with modern extraction technologies. It has been based on a large number of clinical trials and assessments using various herbal extracts taken from the parent plant in a variety of ways, and mixed together in a variety of different proportions. The components to this invention are listed in Table 1. The preferred proportions of the invention are listed in Table 2, and the proportions in a particularly preferred iteration of the invention are listed in Table 3. The composition of the invention can be made from herbal preparations of the flowers, roots, bark, stems, and leaves of the herb. In the case of the current invention, herbal preparations were extracted from the herbs found in Table 1, then mixed in a variety of formulations which were then tested for effectiveness, with the resulting preferred and particularly preferred formulations appearing in Tables 2 and 3 respectively. This data, along with the scientific data presented herein, provides adequate detail for one skilled in the art to prepare and administer the invention as desired to treat erectile dysfunction and as directed herein, thereby practicing the dietary and therapeutic methods taught by this patent. The exact proportions of the starting quantities of each herb which comprises the invention will depend upon the concentration of the active ingredients which are intended for extraction, and may vary depending on the location, growing conditions, and time of harvest of each herb. Using the methods and proportions as laid out in this patent, a skilled practitioner with a basic knowledge of Chinese herbs, drug preparation, extraction techniques, and pharmacology, should be able to practice this invention in the manner set forth.

Recommended human dosage of this invention is one or two tablets (each containing 300 mg of the invention) per day in cases where erectile dysfunction is an ongoing issue. The patient is advised to continue this regimen along with proper diet and exercise. The regimen is also suggested as appropriate for aging men who do not have symptoms of erectile dysfunction to avoid the onset of erectile dysfunction. No matter what the suggested use of the invention is determined, it is essential that the user also maintain a diet adequate to accomplish the physical changes brought about by this invention.

The herbal compositions and extracts which comprise the substance portion of this invention can be administered orally and can be injected, used as suppositories, or prepared in dry or liquid from for addition to foods and drinks, or as a dry powder which can be mixed with dried drink concentrates such as dried drink mixes, hot chocolate, and instant coffee. It is also contemplated that the herbal substances and extracts could also be produced and sold as a dietary supplement or as a medicinal preparation or compounded with non-toxic pharmaceutically acceptable carriers, such as talc, water, glucose, lactose, gum acacia, gelatin, mannitol, starch paste, magnesium trisilicate, corn starch, keratin, colloidal silica, potato starch, urea and other carriers, in the manufacture of tablets, pellets, capsules, suppositories, solutions, emulsions, suspensions, and other forms. It is also contemplated that additional components of the invention may include auxiliary stabilizing, thickening and/or coloring agents and perfumes.

The manufacture of solid compositions such as tablets or capsules can encompass mixing the extracts or herbal active ingredients with a pharmaceutical carrier (e.g., conventional tableting ingredients such as corn starch, lactose, sucrose, sorbitol, talc, stearic acid, magnesium stearate, dicalcium phosphate or gums) and/or pharmaceutical diluents (e.g., water) to form a solid composition containing a substantially homogeneous mixture of the components of the invention as described in this patent, where the active ingredients are dispersed evenly throughout the mixture such that it can be conveniently and easily “cut” into individual dosages of the invention, including but not limited to coated and non-coated pills, tablets and capsules, liquids, and substance forms appropriate for injection.

The liquid forms of the invention are contemplated to potentially include carrier and/or dilutant aqueous solutions of flavored syrups, aqueous or oil suspensions, and flavored emulsions with edible oils such as cottonseed oil, sesame oil, coconut oil, or peanut oil as well as elixirs and similar pharmaceutical vehicles along with pharmaceutically acceptable dispersing and/or suspending agents including but not limited to synthetic natural gums, such as tragacanth, acacia, alginate, dextran, sodium carboxymethyl cellulose, methylcellulose, polyvinylpyrrolidone or gelatin. One skilled in the pharmaceutical arts will be able to formulate an appropriate mixture based on these guidelines and suggestions. Liquid forms of the invention intended for oral use may be presented as a dry product for reconstitution with water or other suitable vehicles before use, or may be enclosed in a capsule as a liquid.

What I claim is:

1. A composition of matter for treating or reducing the risk of erectile dysfunction, comprising: Herba cynomorii, Rhizhomnas atractylodis macrocephalae, Radix rehmannia glutinosea longui, Herba epimedii, Fructus lycii, Fructus schisandrae chinensis, Radix poloygoni multiflor, Cortex cinnamonia cassiae, Fructus amoni, and Radix ginseng.

2. The composition of claim 1, comprising an aqueous or alcohol or aqueous-alcohol extract of each included herbal component.

3. The composition of claim 2, comprising an extract of the following components in the approximate percentages, shown as ranges, as dry weight: Herba cynomorii (35-45%), Rhizhomnas atractylodis macrocephalae (8-13%), Radix rehmannia glutinosea longui (8-13%), Herba epimedii (8-13%), Fructus lycii (3-8%), Fructus schisandrae chinensis (3-7%), Radix poloygoni multiflor (3-7%), Cortex cinnamonia cassiae (5-10%), Fructus amoni (2-5%), and Radix ginseng (4-8%).

4. The composition of claim 3, further comprising a pharmaceutically acceptable carrier, diluent or additive.

5. The composition of claim 4 where the composition can be marketed and sold as a dietary supplement.

6. The composition of claim 2; comprising an extract of the following components in the approximate percentages, as dry weight: Herba cynomorii (40%), Rhizhomnas atractylodis macrocephalae (10%), Radix rehmannia glutinosea longui (10%), Herba epimedii (10%), Fructus lycii (5%), Fructus schisandrae chinensis (4%), Radix poloygoni multiflor (5%), Cortex cinnamonia cassiae (7%), Fructus amoni (3%), and Radix ginseng (6%).

7. The composition of claim 6, further comprising a pharmaceutically acceptable carrier, diluent or additive.

8. The composition of claim 7, where the composition can be marketed and sold as a dietary supplement.

9. A composition of matter comprising therapeutically effective amounts of the following herbal and other components: Herba cynomorii, Rhizhomnas atractylodis macrocephalae, Radix rehmannia glutinosea longui, Herba epimedii, Fructus lycii, Fructus schisandrae chinensis, Radix poloygoni multiflor, Cortex cinnamonia cassiae, Fructus amoni, and Radix ginseng, where the herbal and other components are mixed in such amounts as to be effective in a mammal for treating or reducing the risk of erectile dysfunction in a mammal.

10. The composition of matter in claim 9, where the mammal is a human being.

11. A method of treating erectile dysfunction, comprising the administration of a form of the invention used in the method comprises extracts of the following plants: Herba cynomorii, Rhizhomnas atractylodis macrocephalae, Radix rehmannia glutinosea longui, Herba epimedii, Fructus lycii, Fructus schisandrae chinensis, Radix poloygoni multiflor, Cortex cinnamonia cassiae, Fructus amoni, and Radix ginseng.

12. The method of claim 12, where, the form of the invention used in the method further comprises an aqueous or alcohol or aqueous-alcohol extract of each included herbal component.

13. The method of claim 12, where, the form of the invention used in treating the erectile dysfunction comprises an aqueous extract of the following components in the approximate percentages, shown as ranges, as dry weight: Herba cynomorii (35-45%), Rhizhomnas atractylodis macrocephalae (8-13%), Radix rehmannia glutinosea longui (8-13%), Herba epimedii (8-13%), Fructus lycii (3-8%), Fructus schisandrae chinensis (3-7%), Radix poloygoni multiflor (3-7%), Cortex cinnamonia cassiae (5-10%), Fructus amoni (2-5%), and Radix ginseng (4-8%).

14. The method of claim 13, where the form of the invention used further comprises a pharmaceutically acceptable carrier, diluent or additive.

15. The method of claim 13, where the method of treatment involves the provision and administration of the forms of the invention used of claim 13.

16. The method of claim 13, where the form of the invention used comprises an extract of the following components in the approximate percentages, as dry weight: Herba cynomorii (40%), Rhizhomnas atractylodis macrocephalae (10%), Radix rehmannia glutinosea longui (10%), Herba epimedii (10%), Fructus lycii (5%), Fructus schisandrae chinensis (4%), Radix poloygoni multiflor (5%), Cortex cinnamonia cassiae (7%), Fructus amoni (3%), and Radix ginseng (6%).

17. The method of claim 16, where the pill further comprises a pharmaceutically acceptable carrier, diluent or additive.

18. The method of claim 16, where the method of treatment involves the provision and, administration of the forms of the invention used of claim 13.

19. The composition of claim 16, where the form of the invention used can be marketed and sold as a dietary supplement.
